(19) United States
(12) Patent Application Publication
(10) Pub. No.: US 2007/0255300 A1
(43) Pub. Date: Nov. 1, 2007

(54) LOOSELY COUPLED LANCET

(75) Inventors: Brian D. VANHIEL, Smyrna, GA (US); Bradley S. KOEPPEL, Smyrna, GA (US); William E. CONWAY, Smyrna, GA (US); Christopher J. RUF, Marietta, GA (US); John C. IRWIN, Woodstock, GA (US); Avi M. ROBBINS, Atlanta, GA (US); Ray LATHROP, Atlanta, GA (US)

Correspondence Address: GARDNER GROFF GREENWALD & VILLANUEVA. PC, 2018 POWERS FERRY ROAD, SUITE 800, ATLANTA, GA, 30339, US

(73) Assignee: FACET TECHNOLOGIES, LLC, Marietta, GA (US)

(21) Appl. No.: 11/619,436

(22) Filed: Jan. 3, 2007

Related U.S. Application Data
(63) Continuation-in-part of application No. 10/505,352, filed on Aug. 19, 2004.
(63) Continuation-in-part of application No. 10/987,591, filed on Nov. 12, 2004.

(51) Int. Cl. A61B 5/151

(52) U.S. Cl. 606/181

(57) ABSTRACT

A low-pain lancing device includes a drive mechanism for loosely engaging a lancet and driving the lancet along an axial path. The drive mechanism has a first coupling element and the lancet has a second coupling element, which together form a loose coupling therebetween, wherein interaction between the first and second coupling elements permits a degree of freedom of movement between the lancet and the drive mechanism in a lateral direction, perpendicular to the axial path of travel of the lancet, to prevent non-axial movement of the lancet tip during lancing and thereby reduce pain experienced by the subject.

### CROSS-REFERENCE TO RELATED APPLICATIONS

This application is a continuation-in-part of and claims priority benefit to U.S. patent application Ser. No. 10/505,352 filed Aug. 19, 2004, and is a continuation-in-part of and claims priority benefit to U.S. patent application Ser. No. 10/987,591 filed Nov. 12, 2004; both of which are incorporated herein by reference in their entireties.

### TECHNICAL FIELD

The present invention relates generally to medical devices, and more particularly to low-pain lancing devices for medical diagnostic sampling.

### BACKGROUND OF THE INVENTION

Lancing devices are commonly used to penetrate the skin of a subject and obtain a sample of blood or other body fluid, as in the testing of blood sugar levels by diabetics. Typically, a lancet having a sharp point is translationally mounted within a lancing device. The lancet is driven by a spring or other biasing means to cause the sharp point to extend a small distance through an opening in the housing of the lancing device and into the subject's skin, creating a wound from which the sample of body fluid is collected.

It has been discovered that the drive mechanisms of many previously known lancing devices impart off-axis motion to the lancet, causing the lancet tip to move laterally and/or transversely relative to the longitudinal axis of the lancet. Even small amounts of off-axis vibration or misalignment can cause tearing or pulling of the skin during the lancing sequence, increasing pain to the subject. Decreasing the pain experienced during lancing generally leads to greater product acceptance and improved compliance with a prescribed testing regimen, and is therefore a substantial advantage.

Attempts have been made to reduce off-axis movement of the lancet tip by tightly coupling the lancet to the drive mechanism and placing tightly toleranced guidance features on the driving mechanism. A significant disadvantage of this approach is that any off-center movement or angulation of the drive mechanism translates directly into the lancet. Another disadvantage is that any vibrations introduced by the drive springs and return springs of the lancing device tend to be communicated to the lancet, potentially leading to increased pain to the subject.

Thus it can be seen that needs exist for improvements to lancing devices to provide reduced off-axis movement of the lancet tip imparted by the drive mechanism of a lancing device, and thereby reduce the pain experienced by the subject during use. It is to the provision of an improved lancing device and lancet meeting these and other needs that the present invention is primarily directed.

### SUMMARY OF THE INVENTION

In example forms, the lancing device and lancet of the present invention reduce pain by eliminating or minimizing non-axial movement and misalignment of the lancet during a lancing procedure. The lancet is not tightly coupled to the moving parts of the driving mechanism, but rather is loosely coupled thereto. In this manner, the lancet coupling of the present invention provides one or more degrees of freedom of motion between the lancet and the drive mechanism of the lancing device. This coupling mechanism and method preferably pulls and pushes the lancet along a linear path coincident with the longitudinal axis of the lancet needle, preferably using a single point of contact in either direction. The lancet is preferably further guided by a smooth non-moving track or other guidance feature, to produce a straighter and smoother lancet travel throughout the lancet stroke. This allows the lancet to stay on axis and not be affected by vibrations and off-axis travel of the drive mechanism. The lancet of the present invention therefore projects into and out of the skin along a straight linear path, without significant lateral movement. Vibrations and misalignment from the driver, piston, or springs of the lancing device are not directly transferred to the lancet. As a result, a much smoother and straighter lancet travel into and out of the skin is achieved, leading to reduction in pain experienced by the subject.

In one aspect, the present invention is a lancing device including a drive mechanism for loosely engaging a lancet and driving said lancet along an axial path. The drive mechanism includes a first coupling element and the lancet includes a second coupling element, wherein interaction between the first and second coupling elements permits a degree of freedom of movement in a lateral direction, perpendicular to the axial path, between the lancet and the drive mechanism.

In another aspect, the invention is a low-pain lancing device. The lancing device includes a guidepath for constraining motion of a lancet to a defined path of travel, and a drive mechanism including a loose coupling transferring force to the lancet in a first direction along the defined path of travel, but not transferring force to the lancet in a second direction out of the defined path of travel.

In still another aspect, the invention is a lancing device including a drive mechanism comprising a lancet carrier having a first coupling member; a lancet having a sharp tip, a second coupling member for engagement with the first coupling member, and a guide surface; and at least one fixed guidance element for cooperation with the guide surface of the lancet to define a path of travel of the lancet. The first and second coupling members are loosely coupled to permit relative motion therebetween in a direction perpendicular to the path of travel of the lancet.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of a lancet and a portion of the drive mechanism of a lancing device according to an example form of the invention.

FIG. 2 is a perspective view of a lancet and a portion of the drive mechanism of a lancing device according to another example form of the invention.

FIGS. 3a and 3b are perspective views of a lancet and a portion of the drive mechanism of a lancing device according to still another example form of the invention, shown in a disengaged configuration (FIG. 3a) and an engaged configuration (FIG. 3b).

FIG. 4 shows a lancet-receiving portion of the drive mechanism of a lancing device according to another example form of the invention.

FIG. 5 shows a lancet-receiving portion of the drive mechanism of a lancing device according to still another example form of the invention.

FIG. 6 shows a lancet-receiving portion of the drive mechanism of a lancing device according to another example form of the invention.

FIG. 7 shows a lancet and a portion of the drive mechanism of a lancing device according to another example form of the invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

With reference now to the drawing figures, the lancing device of the present invention provides smooth and straight lancet travel by loosely coupling the lancet with the drive mechanism of a lancing device. In typical lancing devices, the drive mechanism is acted upon by many different sources including the drive spring, the return spring, the depth adjust, and the firing button. Each of these can have a different effect on the travel of the drive mechanism and can cause it to vibrate or rotate off center from intended path of travel. The lancing device of the present invention allows the lancet to travel freely down a non-moving, smooth and straight track, sleeve, cylinder, channel or other guidepath, with a linear push and pull application of driving force applied from the drive mechanism to the lancet. Little or no off-axis movements or vibrations are translated into the lancet, thereby reducing pulling or tearing of the skin at the lancing site, and accordingly reducing the degree of pain experienced by the subject.

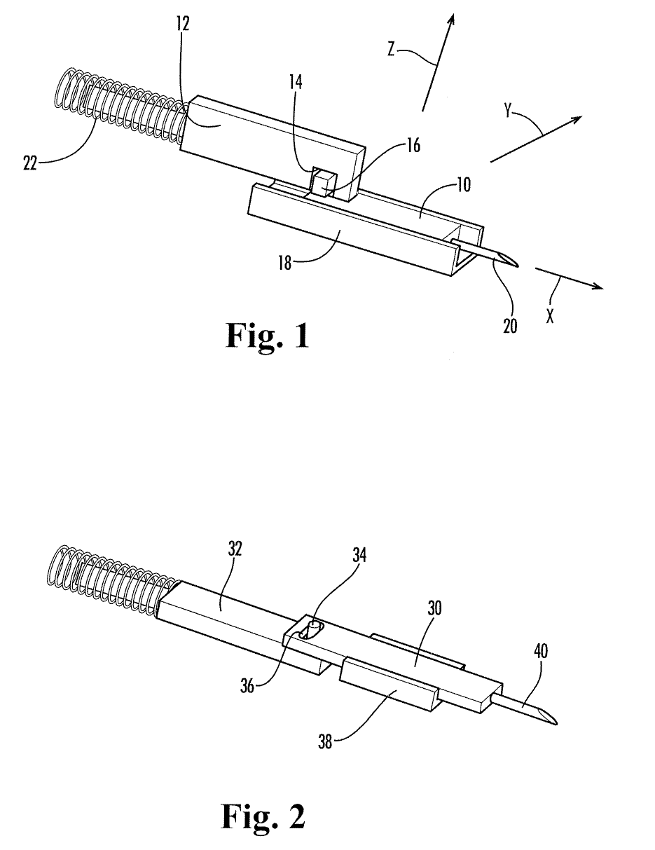

FIG. 1 shows a loosely coupled lancet 10 according to an example embodiment of the invention. A lancet carrier 12 of the drive mechanism of a lancing device includes a slot or channel 14, which hooks around and releasably engages a rib or foot portion 16 of the lancet 10. A fixed guide track or channel portion 18 of the lancing device engages at least one guide surface portion of the lancet 10 with a smooth running tolerance, and defines a guidepath constraining the lancet to a linear path of travel in alignment with the axis X of the lancet needle 20. The sharp tip of the lancet needle 20 is optionally protected from inadvertent contact by a removable sterility cap or cover. One or more biasing members 22, such as a drive spring, return spring, electromechanical actuator, or other propulsion means drive the lancet along a path of travel whereby the lancet needle 20 projects outwardly of the lancing device housing to pierce the subject's skin at the lancing site for sampling of blood or other body fluid. The slot or channel 14 of the lancet carrier 12 is preferably slightly wider than the rib or foot portion 16 of the lancet 10 in the direction of axis X, providing a first degree of freedom of movement therebetween in the axial or X direction. The rib or foot portion 16 of the lancet 10 preferably slides freely side-to-side in the slot or channel 14 of the lancet carrier 12, providing a second degree of freedom of movement in the lateral or Y direction; and more preferably also slides freely in the up-and-down direction, providing a third degree of freedom of movement in the transverse or Z direction (X, Y and Z-axes being defined in mutually perpendicular dimensions). In this manner, a substantially one-dimensional, linear force vector is applied to the lancet by the drive mechanism of the lancing device along both the forward and return portions of the lancing stroke, and any off-axis motion or vibrations generated by the drive mechanism of the lancing device are not transferred to the lancet.

FIG. 2 shows a loosely coupled lancet 30 according to another embodiment of the invention. The lancet carrier 32 of the lancing device's drive mechanism is connected to the lancet 30 by a pin portion 34 of the lancet carrier, which is engaged within a slot 36 formed in the lancet. In alternate forms of the invention, the male and female portions of the loose coupling are arranged in an inverse configuration, with the pin extending from the lancet and the slot being formed in the carrier. The pin-and-slot coupling permits application of drive force to the lancet only in a direction perpendicular to the slot, thereby minimizing non-axial forces acting on the lancet. The slot 36 preferably has a greater dimension than the pin 34 in at least one of the axial and lateral directions, and preferably greater dimensions in both the axial and lateral directions, thereby allowing relative motion between the lancet and the carrier in one or both of the axial and lateral directions. The pin-and-slot coupling preferably also permits relative motion between the lancet and the carrier in the transverse direction. A guide track or channel 38 constrains the lancet to travel along a linear path aligned with the axis of the lancet needle 40. One or both lateral side faces and/or one or both transverse side faces of the lancet body act as guide surfaces for sliding engagement along the guidepath defined by the guide track or channel 38. The loose coupling between the lancet and the carrier preferably allows relative motion therebetween in a direction generally perpendicular to a line of contact between the guidepath of the lancing device and the guide surface(s) of the lancet.

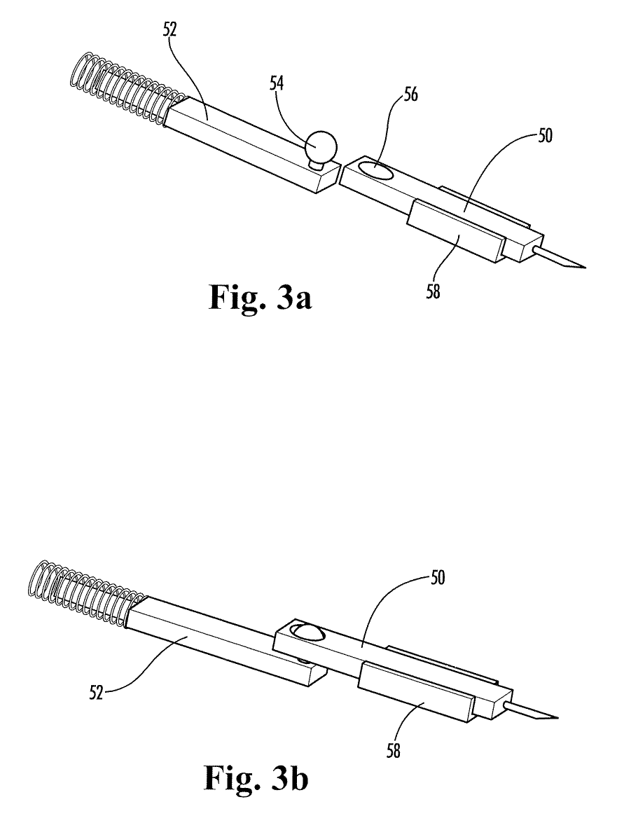

FIGS. 3a and 3b show a loosely coupled lancet 50 according to still another embodiment of the invention, wherein the lancet carrier 52 of the lancing device's drive mechanism and the lancet are connected by a ball-and-socket coupling. In the depicted embodiment, the ball 54 projects from the carrier and the socket 56 is formed in the lancet. Alternate forms of the invention may incorporate the inverse configuration, with the ball projecting from the lancet and the socket being formed in the carrier. The exterior diameter of the ball is preferably sufficiently less than the interior diameter of the socket to permit a degree of freedom of motion or “play” in the coupling in the axial, lateral and transverse directions, and to provide single points of contact between the components of the coupling for applying a linear motive force for propulsion of the lancet along the forward and return portions of the lancing stroke, so that non-axial forces are not imparted on the lancet during firing and retraction. The lancet is guided along its linear path by a guide channel 58 or other guidance member of the lancing device.

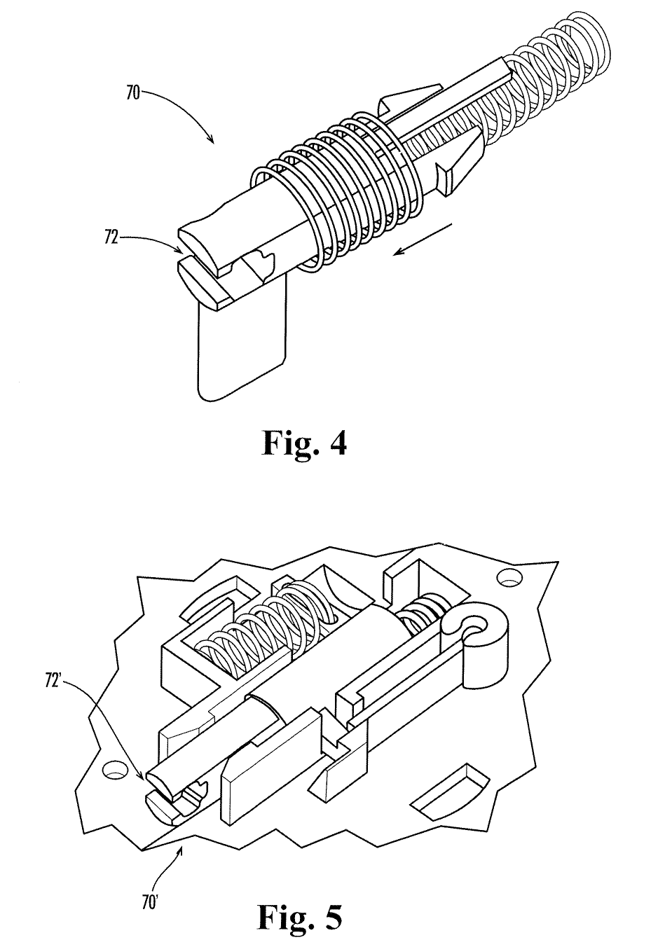

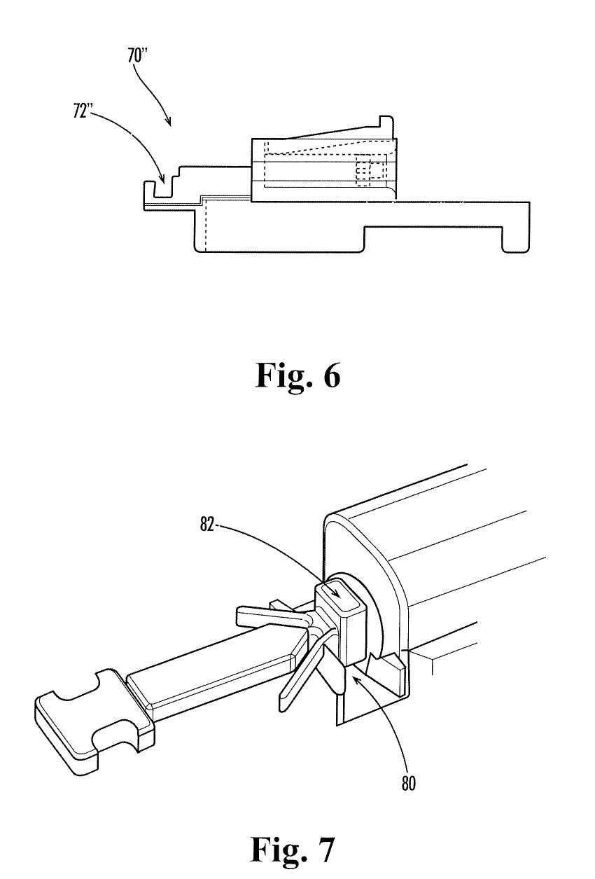

FIGS. 4, 5 and 6 respectively show further embodiments of lancet carrier portions 70, 70′, 70″ of a lancing device for loosely coupling with a cooperating portion of a lancet, within the scope of the present invention. Lancet carrier portions 70, 70′ comprise channels 72, 72′ extending laterally through the carrier and bounded on the top and bottom, which permit a degree of freedom of motion of the lancet in at least the lateral direction relative to the carrier, and thereby prevent introduction of off-axis force or vibration to the lancet in at least the lateral direction. The lancet carrier 70″ of FIG. 6 comprises an open-topped channel 72″ extending laterally through the carrier, which permits motion of the lancet in at least the lateral (side-to-side) and transverse (up and down) directions relative to the carrier, and thereby prevents introduction of non-axial motion and vibrations to the lancet. FIG. 7 shows a similar open-topped channel of a lancet carrier 80, loosely coupled with a rear fin or flange 82 of a cooperating lancet.

While the invention has been described with reference to preferred and example embodiments, it will be understood by those skilled in the art that a variety of modifications, additions and deletions are within the scope of the invention, as defined by the following claims.

What is claimed is:

1. A lancing device comprising a drive mechanism for loosely engaging a lancet and driving said lancet along an axial path, said drive mechanism comprising a first coupling element and said lancet comprising a second coupling element, wherein interaction between the first and second coupling elements permits a degree of freedom of movement in a lateral direction, perpendicular to the axial path, between the lancet and the drive mechanism.

2. The lancing device of claim 1, wherein the first and second coupling elements also permit a degree of freedom of movement between the lancet and the drive mechanism in a transverse direction, perpendicular to the axial path and the lateral direction.

3. The lancing device of claim 1, wherein the first and second coupling elements also permit a degree of freedom of movement between the lancet and the drive mechanism in the direction of the axial path.

4. The lancing device of claim 1, wherein the first and second coupling elements permit a degree of freedom of movement between the lancet and the drive mechanism in a transverse direction, perpendicular to the axial path and the lateral direction, and a degree of freedom of movement between the lancet and the drive mechanism in the direction of the axial path.

5. The lancing device of claim 1, wherein the lancing device further comprises a guidepath for sliding engagement with a cooperating guide surface of the lancet to define the axial path.

6. The lancing device of claim 1, wherein one of the first and second coupling elements comprises a channel and the other of the first and second coupling elements comprises a rib for engagement within the channel.

7. The lancing device of claim 1, wherein one of the first and second coupling elements comprises a pin and the other of the first and second coupling elements comprises a slot for receiving the pin.

8. The lancing device of claim 1, wherein one of the first and second coupling elements comprises a ball and the other of the first and second coupling elements comprises a socket for receiving the ball.

9. A low-pain lancing device comprising:
a guidepath for constraining motion of a lancet to a defined path of travel; and
a drive mechanism comprising a loose coupling transferring force to the lancet in a first direction along the defined path of travel, but not transferring force to the lancet in a second direction out of the defined path of travel.

10. The low-pain lancing device of claim 9, wherein the loose coupling comprises first and second coupling members permitting relative motion therebetween in the second direction.

11. The low-pain lancing device of claim 9, wherein the first coupling member is a recess, and the second coupling member is a projection movable within the recess.

12. The low-pain lancing device of claim 9, wherein the first coupling member is a channel, and the second coupling member is a rib movable within the channel.

13. The low-pain lancing device of claim 9, wherein the first coupling member is a slot, and the second coupling member is a pin movable within the slot.

14. The low-pain lancing device of claim 9, wherein the first coupling member is a socket, and the second coupling member is a ball movable within the socket.

15. The low-pain lancing device of claim 9, wherein the guidepath comprises a channel extending linearly along the defined path of travel.

16. A lancing device comprising:
a drive mechanism comprising a lancet carrier having a first coupling member;
a lancet comprising a sharp tip, a second coupling member for engagement with the first coupling member, and a guide surface; and
at least one fixed guidance element for cooperation with the guide surface of the lancet to define a path of travel of the lancet;
wherein the first and second coupling members are loosely coupled to permit relative motion therebetween in a direction perpendicular to the path of travel of the lancet.

17. The lancing device of claim 16, wherein the guidance element comprises a first sidewall for engaging a first lateral side face of the lancet, a second sidewall for engaging a second lateral side face of the lancet, and a base panel extending between the first and second sidewalls for engaging a transverse side face of the lancet.

18. The lancing device of claim 16, wherein the path of travel of the lancet is in an axial direction extending along an X-axis, and wherein the first and second coupling members permit relative motion therebetween in a lateral direction along a Y-axis perpendicular to the X-axis.

19. The lancing device of claim 18, wherein the first and second coupling members also permit relative motion therebetween in a transverse direction along a Z-axis perpendicular to the X-axis and the Y-axis.

20. The lancing device of claim 18, wherein the first and second coupling members also permit relative motion therebetween in the axial direction.
